(19) United States
(12) Patent Application Publication
(10) Pub. No.: US 2014/0094917 A1
(43) Pub. Date: Apr. 3, 2014

(54) Expandable fusion cage with cam adjuster

(71) Applicant: Anthony A. Salerni, Bedford, NH (US)

(72) Inventor: Anthony A. Salerni, Bedford, NH (US)

(21) Appl. No.: 13/633,210

(22) Filed: Oct. 2, 2012

(51) Int. Cl. A61F 2/44

(52) U.S. Cl. 623/17.16

(57) ABSTRACT

An expandable fusion cage has an elongated cage body with a central hollow region, a posterior end and an anterior end with first and second anterior end portions in communication with the central hollow region, a first longitudinal member and a second longitudinal space from and substantially parallel to the first longitudinal member extending between the posterior end and the first anterior end portion, and a cam body having a cam surface on a periphery of the cam body and a cam adjusting feature facing the central hollow region where the cam body is positioned between the first anterior end portion and the second anterior end portion and is adapted to rotatably engage the cam surface with the first anterior end portion and the second anterior end portion to change the orientation of the anterior end of the cage body between a collapsed configuration and an expanded configuration.

### BACKGROUND OF THE INVENTION

1. Field of the Invention

The present invention relates generally to fusion cage medical devices. More particularly, the present invention relates to an expandable fusion cage.

2. Description of the Prior Art

A small but significant percentage of the population experiences chronic back pain related to spinal disorders. Commonly, a patient experiences pain caused by degeneration of intervertebral discs, which reside between adjacent vertebrae. Motion in a patient's spine occurs between adjacent vertebrae through the disc, which lies between anterior portions of adjacent vertebrae, and facet joints, which lie on either side of posterior portions of the vertebrae. As the disc degenerates, the nucleus of the disc becomes thinner and less able to accommodate compression between the vertebrae. When the nucleus becomes thinner, the annulus of the disc handles abnormal compression loads and the annulus also is less able to control spinal movement. As a result, the patient may experience pressure on nerves from the disc bulging into the nerve passageways, damage or tears to the disc, and disc herniation. These conditions result in arthritis of the facet joints, back pain, and nerve injury.

To alleviate pain from these conditions, surgeons may fuse together adjacent vertebrae using a bone grafting method. To immobilize the spine and allow adjacent vertebrae to grow together, a hollow spinal “cage” is placed in the disc space between vertebrae on either side of the spinal column after removing part of the disc. Typically, a bone graft material is inserted within the hollow cage to enhance the “growth” of two or more vertebrae together. Taking on one of the disc's functions, the cage maintains the space between vertebrae.

When a cage is inserted posteriorly, it must be sized to fit between the superior and inferior endplates of adjacent vertebrae. These endplates are often not parallel in the lumbar vertebrae due to curvature of the spine, or lordosis. The disc space between vertebrae tapers from the anterior portion to the posterior portion. In other words, the vertical distance between posterior endplates is typically smaller than the vertical distance between anterior endplates. Because the posterior spacing is smaller, the surgeon may have to use a cage with parallel superior and inferior surfaces, which fit loosely at the anterior side and tightly at the posterior side; insert the cage where the patient's spine is not curved; or insert the spinal cage anteriorly. All of these options are non-ideal.

Several variations in spinal cages have been developed in an attempt to overcome the problems with posterior placement of a spinal cage. One expandable intervertebral cage, described in U.S. Pat. No. 6,814,756 to Michelson, has a cylindrical body formed by an upper body portion, and a lower body portion. The upper body portion and lower body portion are hinged at a posterior end. Anterior ends of the upper and lower body portions are spread apart using an extrinsic tool. The upper and lower body portions are then held in an expanded position by positioning a blocker between anterior portions of the upper and lower body portions.

Another spinal cage, described in U.S. Pat. No. 6,491,724 to Ferree, includes either a passive or an active expansion mechanism in a box-like intervertebral cage. Passive expansion mechanisms include making the cage of a material with a shape memory that causes the cage to naturally expand from a compressed state to an expanded state once the cage is positioned within the intervertebral space. An active expansion mechanism has an externally-accessible mechanism that is used to increase the anterior height of the cage once it is positioned within the intervertebral space. One active mechanism has a slug positioned on a threaded rod within the cage. The cage is hinged at its posterior end. The slug moves posterior to anterior when the rod is rotated, causing the upper member of the cage to hinge and move upwardly to assume a wedge-shaped orientation.

Another cage device, described in U.S. Pat. No. 6,443,989 to Jackson, has a box-like body with upper and lower walls. The upper and lower walls are joined at a rear wall that functions as a spring hinge. The upper and lower walls have anterior ends that are supported in a non-expanded configuration by spaced-apart feet that project out from the walls. Received in the rear wall of the cage is an elongate, threaded expansion member that has a head with an anterior wedge portion. The wedge portion engages the anterior ends of the walls and forces the walls apart as the expansion member is screwed into the body to advance the wedge portion towards the anterior ends.

### SUMMARY OF THE INVENTION

Currently-available devices described above exhibit various disadvantages. Cylindrical devices require a reamed channel in the vertebral bone between vertebrae, which weakens the bone. Also, other cylindrical devices open radially at the anterior end of the fusion cage, which is undesirable if lateral expansion is not needed. Existing rectangular cages have a piston-type mechanism extending through the cage body to cause the upper and lower portions of the device to expand. The action of the piston-like mechanism on the unsecured cage forces the device out of position in the direction of the piston's movement. Therefore, what is needed is an improved expandable fusion cage for expanding the anterior portion of the cage.

It is an object of the present invention to provide an expandable fusion cage device for posterior placement between adjacent vertebrae.

It is another object of the present invention to provide an expandable fusion cage that may be adjusted from a collapsed position to an expanded position.

It is another object of the present invention to cause expansion of the fusion cage without using a piston-like device that extends through the cage body.

It is another object of the present invention that the anterior cage expansion occurs by adjusting the device from the posterior end of the fusion cage.

The present invention achieves these and other objectives by providing an expandable fusion cage with a cam adjuster. In one embodiment, the expandable fusion cage has an elongated cage body with a posterior end, an anterior end, and a central hollow region extending along a central longitudinal axis of the cage body. The posterior end has an access opening therethrough in communication with the hollow region. The anterior end has a first anterior end portion and a second anterior end portion. A first longitudinal member extends between the posterior end and the first anterior end portion substantially parallel to the central longitudinal axis. A second longitudinal member extends between the posterior end and the second anterior end portion substantially parallel to the central longitudinal axis. A cam body has a cam surface on a periphery of the cam body and a cam adjusting feature facing the central hollow region where the cam body is positioned between the first anterior end portion and the second anterior end portion. The cam body is adapted to rotatably engage the cam surface with the first anterior end portion and the second anterior end portion to change the orientation of the anterior end of the cage body between a collapsed configuration and an expanded configuration, where the cam body has at least one engagement feature for rotating the cam body.

In another embodiment of the present invention, the cam body is substantially oblong.

In another embodiment of the present invention, the cam adjusting feature includes one of more structures such as a recessed structure or a protruding structure.

In another embodiment of the present invention, the protruding structure has a rectangular adjustment member and a cam shaft, where the cam shaft extends substantially parallel to the central longitudinal axis of the elongated cage body and is fixedly attached between the cam body and the rectangular adjustment member.

In another embodiment of the present invention, the first anterior end portion and the second anterior end portion is in a collapsed configuration and defines an open region therebetween. The open region extends along the central longitudinal axis at least partially through at least one of the first anterior end portion and the second anterior end portion. The open region is shaped and configured for rotatable interaction with the cam body.

In another embodiment of the present invention, the open region also includes at least one locking notch configured to receive an end of the cam body.

In another embodiment of the present invention, at least one of the first anterior end portion and the second anterior end portion has an end wall adjacent to the open region and extending substantially perpendicular to the central longitudinal axis.

In another embodiment of the present invention, the cam body includes a cam retainer configured to retain the cam body between the first anterior end portion and the second anterior end portion.

In another embodiment of the present invention, the first elongated member and/or the second elongated member includes a pair of spaced-apart members.

In another embodiment of the present invention, one or both of the first longitudinal member and the second longitudinal member is hingedly attached to the posterior end.

In another embodiment of the present invention, the first longitudinal member and the second longitudinal member are fixedly attached to the posterior end. One of the posterior end or a junction between the posterior end and each of the first and second longitudinal members is bendable to permit the anterior end of the fusion cage to convert from a collapsed configuration to an expanded configuration upon rotation of the cam body.

In another embodiment of the present invention, the cage body is made of a biocompatible material.

### BRIEF DESCRIPTION OF THE DRAWINGS

FIG. 1 illustrates a posterior perspective view of one embodiment of the present invention showing a cage in an expanded position and a cam adjustment assembly.

FIG. 2 illustrates a posterior perspective view of the cage of FIG. 1 shown in a closed position.

FIG. 3 illustrates a side view of the cage of FIG. 1 in a closed position.

FIG. 4 illustrates an anterior end view of the cage shown in FIG. 1.

FIG. 5 illustrates a posterior perspective view of an embodiment of a cage in an expanded position and showing posterior end portions with recesses therein.

FIG. 6 illustrates a side view of the cage of FIG. 5 in an expanded position and shows surfaces of posterior end portions of the cage.

FIG. 7 illustrates an anterior end view of the cage of FIG. 5 in a closed position and shows anterior end portions and recesses therein.

FIG. 8 illustrates an outline of an embodiment of the open area defined by recesses in upper and lower anterior end portions of the cage of FIG. 5 in a closed position.

FIG. 9 illustrates another embodiment of anterior end portions of a cage in an expanded position and shows slot-like recesses in the end portions.

FIG. 10 illustrates cross-sectional views of one embodiment of an anterior end of a cage and cam body of the present invention showing the anterior end in (a) a collapsed configuration, (b) an intermediate expanded configuration, and (c) a further expanded configuration with a cam body locked in position.

FIG. 11 illustrates a perspective exploded view of one embodiment of a cam adjuster assembly that includes a cap, a cam body, a cam shaft, and an adjustment member.

FIG. 12 illustrates a perspective view of the embodiment of the cam adjustment assembly of FIG. 10 as assembled.

FIG. 13 illustrates a perspective view of an alternate embodiment of a cam body with at least one engagement feature.

### DETAILED DESCRIPTION OF THE PREFERRED EMBODIMENT

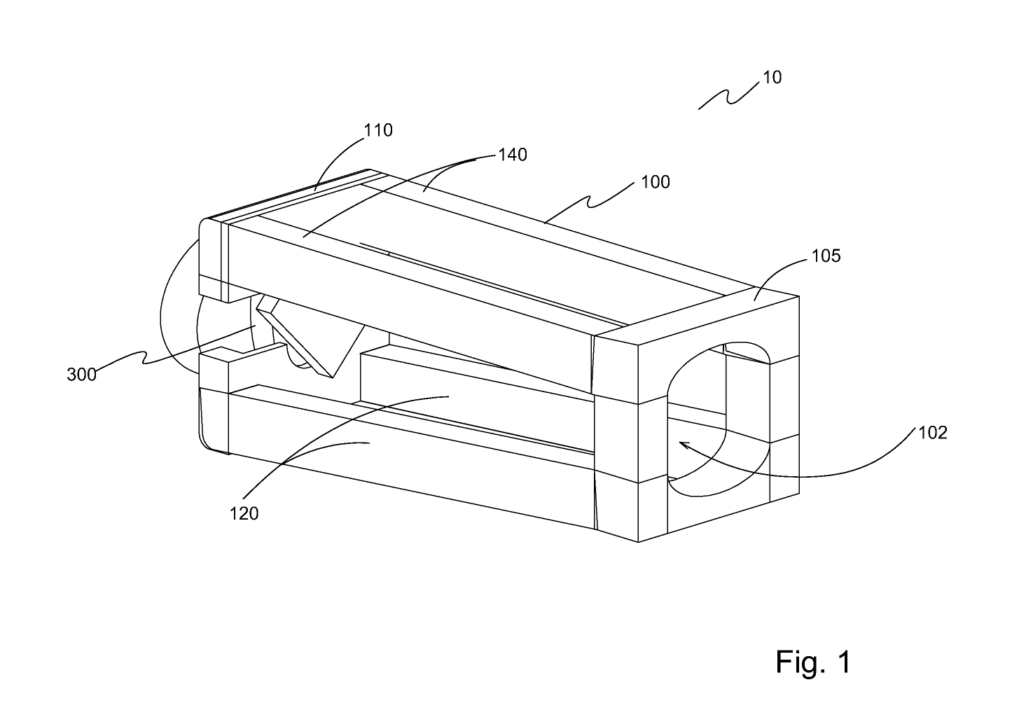

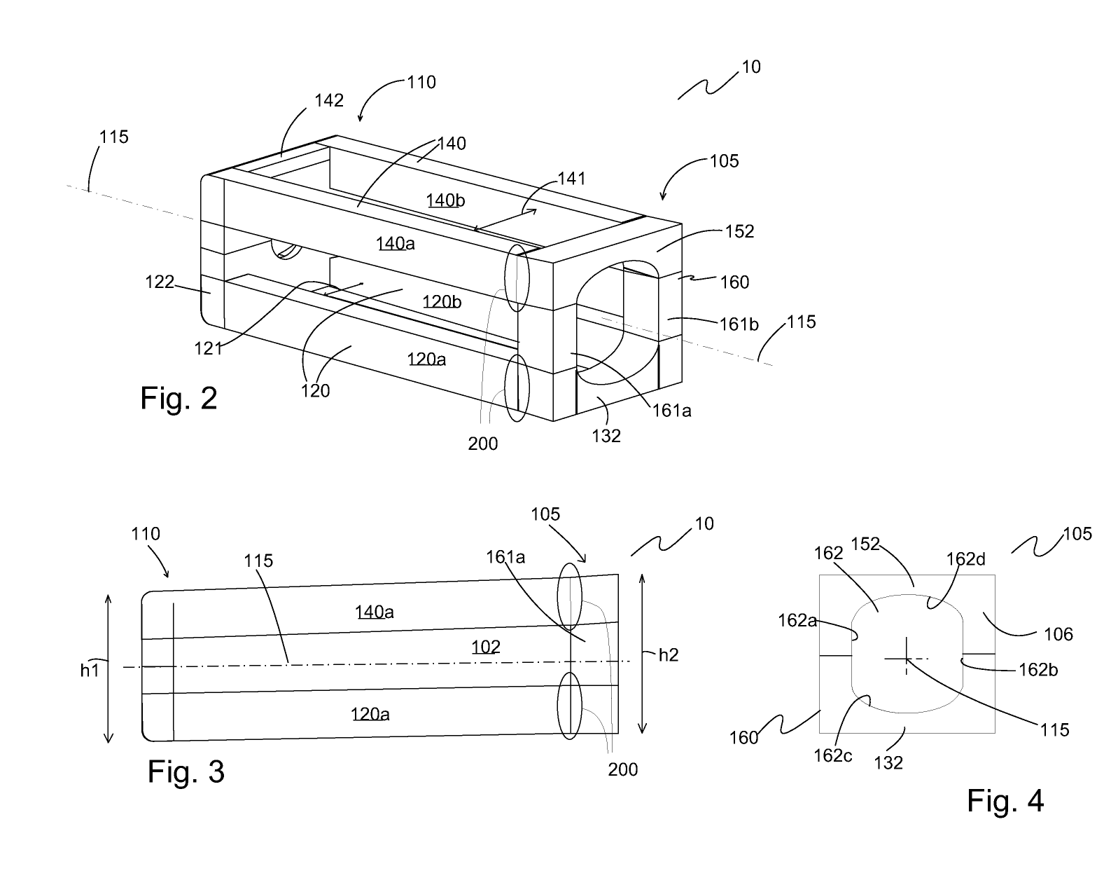

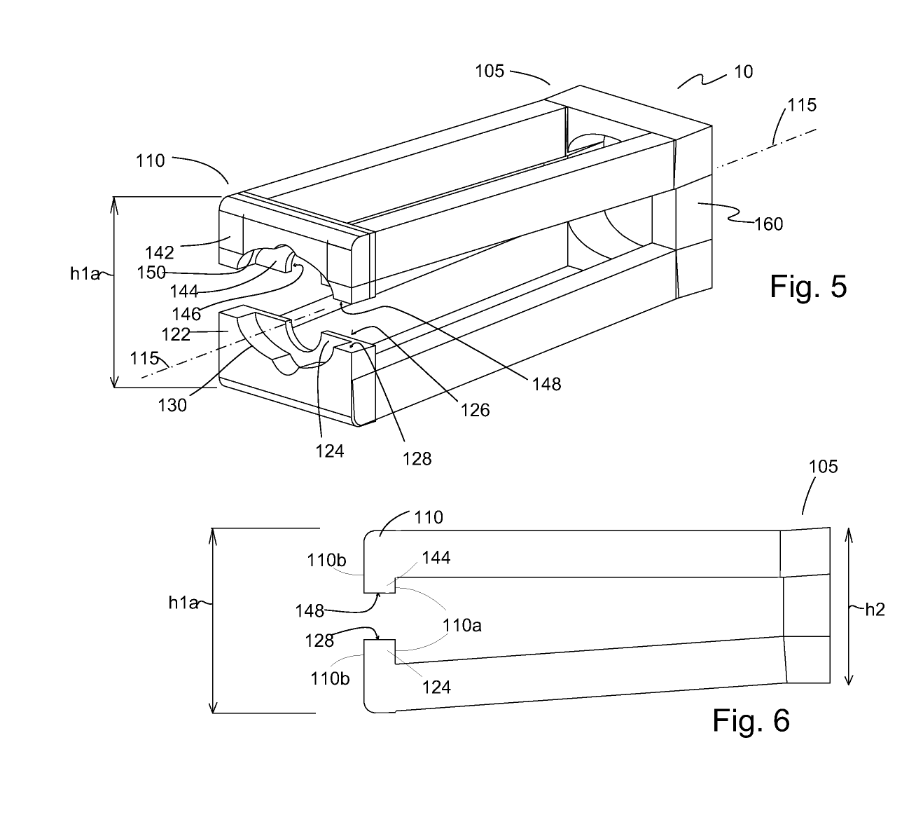

The preferred embodiments of the present invention are illustrated in FIGS. 1-13. FIG. 1 illustrates a perspective view of one embodiment of an expandable fusion cage 10. Expandable fusion cage 10 includes an elongated cage body 100 with a posterior end 105 and an anterior end 110 that defines a central hollow region 102. Fusion cage 10 also includes a cam adjustment assembly 300 positioned to rotatably engage anterior end 110 of cage body 100. Cam adjustment assembly 300 may be rotated to cause cage body 100 to change between a collapsed configuration (shown in FIGS. 2 & 3) and an expanded configuration at anterior end 110 (shown in FIGS. 1, 5 & 6).

FIGS. 2 and 3 illustrate perspective and side views, respectively, of the embodiment of cage body 100 of FIG. 1 in its collapsed configuration. Cage body 100 has a generally-rectangular cross sectional shape that extends between posterior end 105 and anterior end 110 along a central longitudinal axis 115. Cage body 100 has a lower or first longitudinal member 120 with a lower or first anterior end portion 122 and a lower or first posterior end portion 132. Cage also has an upper or second longitudinal member 140 with an upper or second anterior end portion 142 and an upper or second posterior end portion 152. Middle posterior portions 161a, 161b extend between first and second posterior end portions 132, 152, respectively.

In a collapsed configuration, anterior end 110 has a height h1 measured vertically across first anterior end portion 122 and second anterior end portion 142. First anterior end portion 122 may or may not contact second anterior end portion 142 in the collapsed position. Posterior end 105 of cage body 100 has a height h2.

In one embodiment, one or both of first longitudinal member 120 and second longitudinal member 140 are hingedly attached to posterior end 105 of cage body 100. In another embodiment, first posterior end portion 132 and/or second posterior end portion 152 are hingedly connected to each other or to one or more portions 161a, 161b of a posterior body 160 that joins first longitudinal member 120 and second longitudinal member 140.

In another embodiment, first longitudinal member 120 and second longitudinal member 140 are fixedly attached or integral to posterior end 105 of cage body 100. In either embodiment, it is contemplated that a junction 200 between first longitudinal member 120, second longitudinal member 140 and posterior body 160, or posterior body 160, is bendable to permit cage body 100 to convert from a collapsed configuration to an expanded configuration. For example, materials used to construct cage body 100 are preferably sufficiently flexible or thin enough at the bendable junction to permit first and/or second longitudinal members 120, 140 to pivot vertically about posterior end 105 (without requiring a hinge or similar device) to convert cage body 100 between the collapsed configuration and the expanded configuration.

Materials for cage body 100 include biocompatible materials such as, for example, stainless steel, cobalt-chromium-molybdenum alloys, titanium, carbon-reinforced polymers, shape memory alloys, and nylon or other fiber or polymeric materials as described in U.S. Pat. No. 5,192,327, which is incorporated herein by reference in its entirety. Carbon-reinforced polymers include alloys of copper and zinc, nickel titanium, silver and cadmium, and other metals as described in U.S. Pat. No. 5,954,725, which is incorporated herein by reference in its entirety.

In one embodiment, lower or first longitudinal member 120 includes a pair of spaced-apart elongated legs 120a, 120b that extend between first anterior end portion 122 and first posterior end portion 132 of cage body 100. Elongated legs 120a, 120b each extend substantially parallel to central longitudinal axis 115 and are connected at a posterior end 105 by first posterior end portion 132 and at anterior end 110 by first anterior end portion 122. Being spaced apart, elongated legs 120a, 120b define at least one gap or opening 121 between them. Opening 121 is bounded by first anterior end portion 122, first posterior end portion 132, and elongated legs 120a, 120b.

Similarly, second longitudinal member 140 in one embodiment includes a pair of spaced-apart elongated legs 140a, 140b that extend between second anterior end portion 142 and second posterior end portion 152 of cage body 100. Elongated legs 140a, 140b each extend substantially parallel to central longitudinal axis 115 and are connected at posterior end 105 by second posterior end portion 152 and at anterior end 110 by second anterior end portion 142. Elongated legs 140a, 140b define at least one gap or opening 141 between them. Opening 141 is bounded by first anterior end portion 142, first posterior end portion 152, and elongated legs 140a, 140b.

In another embodiment, first longitudinal member 120 and/or second longitudinal member 140 is a generally plate-like or planar structure that extends between first anterior end portion 122 and first posterior end portion 132. In such an embodiment, first longitudinal member 120 preferably has one or more openings 121 and second longitudinal member 140 also preferably has one or more openings 121. Openings 121, 141 allow bone growth between adjacent vertebrae through cage body 100 to bone graft material introduced into a central hollow portion 102 within cage body 100.

FIG. 4 illustrates one embodiment of a posterior end of cage body 100 with a U-shaped first (lower) posterior end portion 132 and a U-shaped second (upper) posterior end portion 152. In a closed position of cage body 100, first posterior end portion 132 and second posterior end portion 152 are opposed to and contact each other to form a substantially rectangular posterior body 160 of cage body 100. Posterior body 160 in some embodiments includes middle portions 161a, 161b between first posterior end portion 132 and second posterior end portion 152 as shown, for example, in FIG. 2. Posterior body 160 has an access opening 162 bounded between first posterior end portion 132 and second posterior end portion 152 and extending along central longitudinal axis 115 through posterior body 160 of cage body 100. In one embodiment, posterior body 160 is a single, unitary component of cage body 100. In other embodiments, posterior body 160 is assembled from multiple components, including first end portion 132 and second end portion 152. In yet other embodiments, posterior body is unitary with first longitudinal member 120 and/or second longitudinal member 140.

Access opening 162 is preferably centered on posterior body 160, but this does not have to be the case. In some embodiments, it may be preferable to position access opening 162 more towards one side of posterior body 160 to permit the user to access the cam adjustment assembly at an angle. In one embodiment, access opening 162 has a generally-oval shape with a pair of opposed straight sides 162a, 162b and a pair of opposed arcuate sides 162c, 162d. Access opening 162 may have other shapes, such as round, rectangular, oval, polygonal, and simple or complex combinations of these shapes so long as the user has sufficient access to the cam adjusting feature 350 to adjust the cam adjustment assembly 300 to convert cage body 100 between a collapsed configuration and an expanded configuration.

FIG. 5 illustrates one embodiment of cage body 100 in an expanded configuration. In an expanded configuration, anterior end 110 has an increased vertical height h1a as compared to height h1 in the collapsed configuration. Height h1a is greater than height h1 due to increased distance between first anterior end portion 122 and second anterior end portion 142. First anterior end portion 122 of cage body 100 has a first end wall 124 with a first inside surface 126 that faces posterior body 160 of cage body 100. Second anterior end portion 142 has a second end wall 144 with a second inside surface 146 that also faces posterior body 160 of cage body 100.

Referring to FIGS. 5 and 6, first end wall 124 has a first wall end surface 128 that faces upward towards a second wall end surface 148 of second end wall 144. Second wall end surface 148 faces downward towards first wall end surface 128. Preferably, first wall end surface 128 and second wall end surface 148 are planar, align with each other, and are capable of contacting each other when cage body 100 is in a collapsed configuration as shown in FIG. 2 for example. First wall end surface 128 and second wall end surface 148 do not have to be planar and instead could also be rippled, angled, arcuate, or have other profiles.

As also shown in FIG. 6, anterior end 110 has an inside anterior surface 110a and an outside anterior surface 110b. Additionally, anterior end 110 is shown in an expanded configuration with an increased vertical height h1a. Conversely, posterior end 105 of cage body 100 has a fixed vertical height h2.

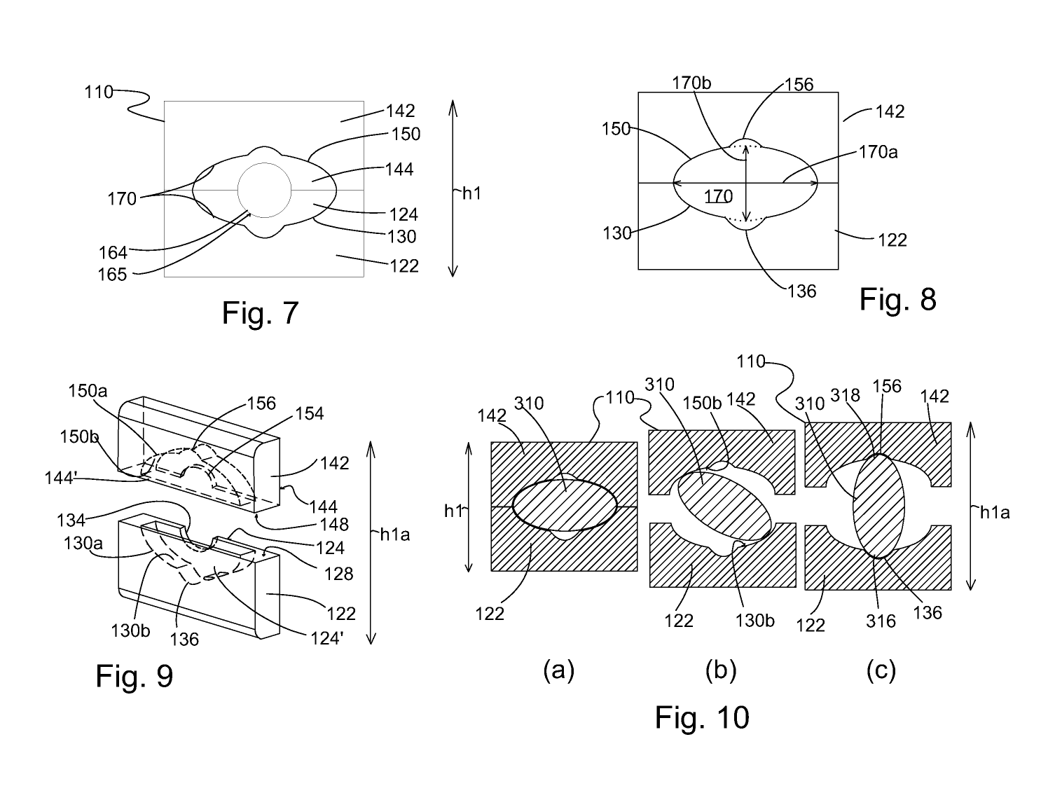

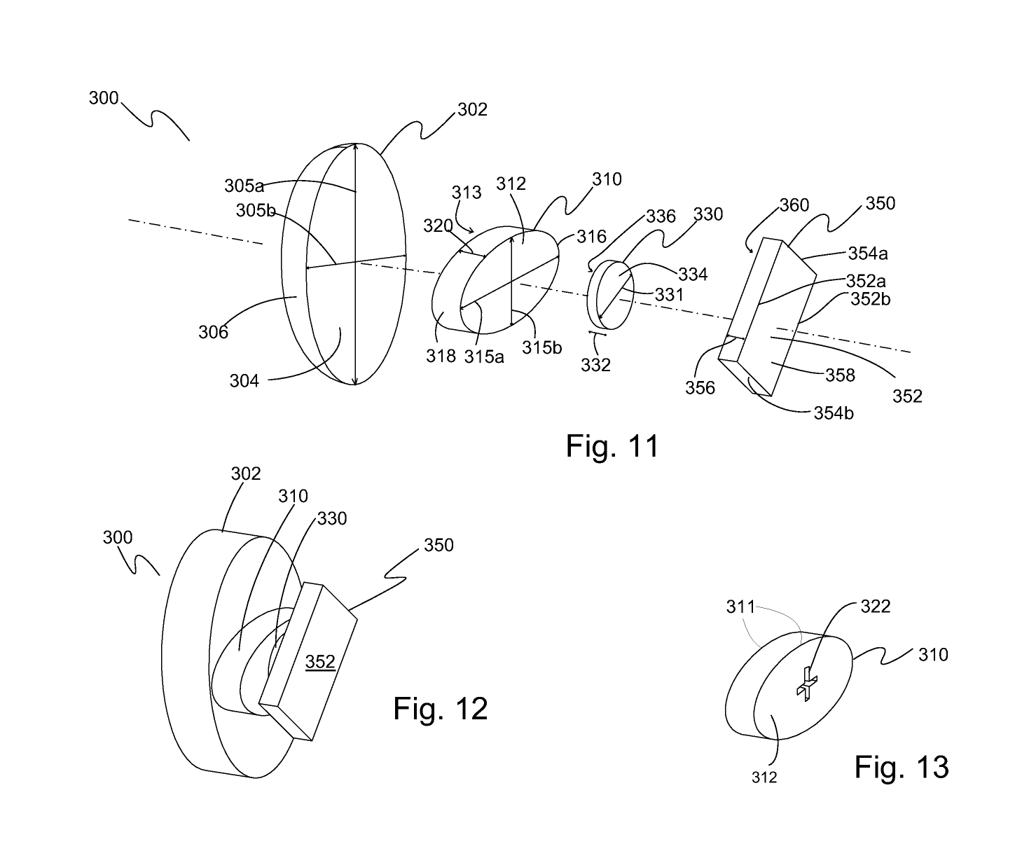

FIG. 7 illustrates an end plan view of an anterior end 110 of cage body 100 in a collapsed configuration. For ease of installing posteriorly in a patient, anterior end 110 has a height h1 that is preferably equal to or smaller than a height h2 of posterior end 105 (shown in FIGS. 3 & 6). In some embodiments, anterior end 110 preferably has one or both of a first cam recess 130 and a second cam recess 150 that extend partially or completely through first and second anterior end portions 122, 124, respectively. In the illustrated embodiment, a first cam recess 130 extends along central longitudinal axis 115 (not shown) partially into first anterior end portion 122 to define a first end wall 124 at an inside anterior end surface 110a (shown in FIG. 6). In another embodiment, first cam recess 130 extends along central longitudinal axis 115 completely through first anterior end portion 122 and having no first end wall 124. The inclusion of first end wall 124 is useful for retaining a cam body 310 within first cam recess 130 by eliminating a separate retaining component on at least one side of cam body 310. Alternatively, first cam recess 130 may extend partially into first anterior end portion 122 from inside anterior surface 110a. In this embodiment (not shown), first end wall 124 is co-planar with outside anterior end surface 110b. In the embodiment where first cam recess extends completely through first anterior end portion 122, an attachment, such as a washer or retaining member, to anterior end 110 of cage body 100 or to one or both of anterior face 313 or posterior face 312 of cam body 310 may be used to maintain the longitudinal position of cam body 310 (shown in FIG. 11) within hollow region 170 between first anterior end portion 122 and second anterior end portion 142. Such an attachment may be, for example, a cam retainer 302 and/or an adjustment member 350, which are discussed more fully below.

Similarly, anterior end 110 preferably has a second cam recess 150 that extends along central longitudinal axis 115 partially or completely through second anterior end portion 142 to define second end wall 144. In one embodiment, second cam recess 150 extends partially into second anterior end portion 142 to define a second end wall 144 at an inside anterior end surface 110a (shown in FIGS. 5 and 6). Second cam recess 150 intersects or extends into second wall end surface 148, which faces downward towards first anterior end portion 122 (better shown in FIG. 9). Alternatively and as described above for first anterior end portion 122, first cam recess 130 may extend partially into second anterior end portion 142 from inside anterior surface 110a. In this embodiment, second end wall 144 is co-planar with outside anterior end surface 110b. In yet another embodiment, second cam recess 150 extends completely through second anterior end portion 142. In this embodiment, an attachment to anterior end 110 of cage body 100 or to one or both of anterior face 313 and posterior face 312 of cam body 310 may be used to maintain the longitudinal position of cam body 310 within hollow region 170 between first anterior end portion 122 and second anterior end portion 142.

As illustrated in FIG. 8, first cam recess 130 and second cam recess 150 preferably each have the shape of one-half of an oval, where bringing first anterior end portion 122 together with second anterior end portion 142 (i.e., contacting first wall end surface 128 with second wall end surface 148) aligns first cam recess 130 with second cam recess 150 to define an open region 170 with a substantially-oval or oblong shape with a major axis 170a and a minor axis 170b. Major axis 170a preferably is positioned along the intersection between first anterior end portion 122 and second anterior end portion 142.

FIG. 9 illustrates another embodiment of anterior end 110 with first and second anterior end portions 122, 142, respectively, shown in an expanded configuration. Anterior end 110 in its expanded configuration has height h1a that is greater than height h1 and height h2 (shown in FIG. 3). In this embodiment, one or both of first cam recess 130 and second cam recess 150 are cam slot recesses 130a and 150a, respectively, that extend substantially perpendicularly to central longitudinal axis 115 into first anterior end portion 122 and/or second anterior end portion 142 through first wall end surface 128 (facing upward) and second wall end surface 148 (facing downward), respectively. Cam slot recesses 130a, 150a each have a cam slot surface 130b, 150b, respectively, that preferably has an arcuate surface that is similar to the shape of cam body 310. In such an embodiment, first cam recess 130 is positioned longitudinally between first end wall 124 and an additional first end wall 124′. Similarly, second cam recess 150 is positioned longitudinally between second end wall 144 and an additional second end wall 144′. Cam slot recess 130a and cam slot recess 150a are thus configured to accept and retain a cam body 310.

In the various embodiments discussed above and as shown in FIG. 9, for example, first cam recess 130 preferably has a sloping or curved first recess surface 130b along the lower boundary of cam slot recess 130a. Similarly, cam slot recess 150a preferably has a sloping or curved second recess surface 150b along the upper boundary of cam slot recess 150a.

In addition to first cam recess 130, first anterior end portion 122 preferably has a first anterior opening 134 extending through first end wall 124 and first wall end surface 128. Similarly, second anterior end portion 142 additionally or alternately has a second anterior opening 154 extending through second end wall 144 and second wall end surface 148. First anterior opening 134 and second anterior opening 154 are preferably semi-circular in shape, but may have other shapes, such as rectangular, curved, polygonal, and combinations of these shapes. It is understood that if other shapes are used, the minor diameter of the shape of first anterior opening 134 and second anterior opening 154 must be larger than a shaft of a cam assembly to allow rotation of a cam shaft 330 (not shown). When cage body 100 is in its collapsed configuration as shown in FIG. 2, first anterior opening 134 and second anterior opening 154 align and define cam shaft opening 164 with diameter 165 (shown in FIG. 7). Cam shaft opening 164 is preferably centered on anterior end 110 of cage body 100, and centered within open region 170.

As illustrated in FIGS. 8 and 9, first cam recess 130 and cam slot recess 130a also preferably include a first locking notch 136 shaped and sized to accept an end or protrusion of cam body 310. First locking notch 136 is an extension of first cam recess 130 and cam slot recess 130a, preferably having a rounded shape corresponding to the shape of an end of cam body 310 and extending into first anterior end portion 122 from an end of minor axis 170b of open region 170. Although not necessary to the functioning of the present invention, second cam recess 150 and cam slot recess 150a preferably include a second locking notch 156 that is shaped and sized to accept an end or protrusion of cam body 310. First locking notch 136 and second locking notch 156 are preferably positioned opposite each other at ends of minor axis 170b of oblong open region 170. Other positions for first and second locking notch 136, 156 are also acceptable.

FIG. 10 illustrates cross-sectional views of first anterior end portion 122, second anterior end portion 142, and cam body 310 in (a) a collapsed configuration, (b) an intermediate expanded configuration, and (c) a further expanded configuration where ends 316, 318 of cam body 310 occupy first locking notch 136 and second locking notch 156, respectively.

As illustrated, cam body 310 is in a horizontal position where anterior end 110 is in a collapsed configuration. In an intermediate expanded configuration as shown in FIG. 10(b), cam body 310 has increased the distance between first anterior end portion 122 and second anterior end portion 142, but is not locked in position. When cam body 310 is rotated, it engages first recess surface 130b and/or second recess surface 150b to increase the vertical distance between first anterior end portion 122 and second anterior end portion 142 of cage body 100. Accordingly, cage body 100 changes from a collapsed configuration with height h1 (as shown, for example, in FIGS. 2-3) to an expanded configuration with height h1a (as shown, for example, in FIGS. 5-6).

In FIG. 10 (c), cam body 310 has been rotated to a vertical position where first end 316 of cam body 310 occupies first locking notch 136 and second end 318 of cam body 310 occupies second locking notch 156 to lock cage body 100 in an expanded configuration.

FIG. 11 illustrates a perspective exploded view of one embodiment of a cam adjustment assembly 300 that includes a cam retainer 302, a cam body 310, a cam shaft 330, and a cam adjusting feature 350. Cam adjusting feature 350 may be a cam body structure 322, a cam adjustment member 352, or the combination of both structures 322, 352. In the illustrated example, cam adjusting feature 350 is a cam adjustment member 352. Cam retainer 302, cam body 310, cam shaft 330, and cam adjustment member 352 are fixedly attached to one another where rotation of cam adjustment member 352 also rotates cam shaft 330, cam body 310, and cam retainer 302. In some embodiments, cam adjustment assembly 300 includes cam body 310 with cam body structure 322, which is discussed in more detail below. Therefore, cam retainer 302 and shaft 330 are optional features.

Cam retainer 302 has a flat posterior retainer face 304 that is preferably round or oval. Retainer face 304 has a diameter or major diameter 305a that is sized to be similar or equal to height h1 of anterior end 110 of cage body 100. Major diameter 305a is greater than any dimension across open region 170 between first anterior end portion 122 and second anterior end portion 142 when cage body 100 is either in a collapsed or expanded configuration. For embodiments where retainer face 304 is oblong, retainer face 304 has a minor diameter 305b. Cam retainer 302 also has a retainer body 306 that is anterior to retainer face 304.

Cam body 310 is preferably oblong in shape and has a posterior face 312 and an anterior face 313 (not visible) on opposite sides of cam body 310. Posterior and anterior faces 312, 313 each have a major diameter 315a and a minor diameter 315b. At the opposite ends of major diameter 315a, cam body 310 has a first end 316 and a second end 318, respectively. Cam body has a thickness 320 between posterior face 312 and anterior face 313. Cam body 310 is sized and shaped to fit within hollow region 170 between first anterior end portion 122 and second anterior end portion 142.

Another embodiment of cam body 310 includes multiple protrusions 322 spaced around its perimeter. The radius to the extent of each protrusion 322 generally increases to enable the user to rotate cam body 310 a small amount (e.g., ⅛ or ¼ of a revolution) to select the amount of expansion at anterior end 110 desired. Rather than having first locking notch 136 and second locking notch 156 on anterior end 110, cam body 310 may optionally include one or more recesses 324 along an outer perimeter surface 119 to lock with a protrusion (not shown) on anterior end 110 of cage body 100 for locking cage body 100 in an expanded position. Protrusions 322 and/or recesses 324 may be of different sizes to provide multiple expanded positions where each expanded position provides a pre-determined vertical separation between first anterior end 122 and second anterior end 142.

Cam shaft 330 is preferably a disc or cylinder that is fixedly attached on its anterior end 336 to posterior face 312 of cam body 310 and on its posterior end 334 to cam adjustment member 352. Shaft 330 has a diameter 331 and a length 332 each sized to allow shaft 330 to extend through cam shaft opening 164 of anterior end 110 of cage body 100. Diameter 331 of shaft 330 is preferably slightly smaller than diameter 165 of cam shaft opening 164 (shown in FIG. 7). The inside surface of cam shaft opening 164 contacts shaft 330 to maintain and guide the position of cam body 310 when cam body 310 is rotated. For smooth rotational operation, shaft 330 preferably has a circular cross sectional shape, but other cross-sectional shapes could also be used. For example, a square or other shape of cam shaft 330 may be used provided that cam shaft opening 164 is sized and configured to accept and allow cam shaft 330 to rotate. In other words, the major diameter of shaft 330 must be smaller than the minor diameter of cam shaft opening 164. In one embodiment, for example, cam shaft 330 has an oblong cross-sectional shape that engages cam shaft opening 164 to increase the vertical separation between first end portion 122 and second end portion 142 in a similar manner as cam body 310 engaging first recess surface 130a.

Cam adjustment member 352 is fixedly connected to posterior end 334 of shaft 330 and resides on the interior of cage body 100 close to or against first inside surface 126 of first end wall 124 and second inside surface 146 of second end wall 144. Cam adjustment member 352 preferably has a substantially flat rectangular shape with a pair of longer sides 352a, 352b, a pair of shorter sides 354a, 354b, and a thickness 356 between a posterior face 358 and an anterior face 360. A rectangular shape allows the user to engage sides of cam adjustment member 352 to rotate it and therefore to rotate cam body 310 whose cam surface 310a on the periphery of cam body 310 engages the corresponding cam surfaces of the anterior end 110. Because expandable spinal cage 10 is positioned within the human body and surrounded by tissue, bone, and the like, a rectangular shape also makes it easier for the user to determine by visual inspection whether the position of cam body 310 corresponds to the collapsed configuration or the expanded configuration of cage body 100.

Cam adjustment member 352 is not limited to the shape of a rectangular block. In other embodiments, cam adjustment member 352 has the general shape and appearance of a flanged nut, such as a flanged hex nut. Rather than having a central threaded opening of a typical flanged hex nut, however, cam adjustment member 352 shaped like a flanged nut is preferably fixedly attached to shaft 330 at its anterior, flanged portion. In this embodiment, the user may engage the sides of the polygonal (e.g., hexagonal) nut portion to rotate cam adjustment member 352 and therefore cam body 310. The round, flanged portion of flanged-nut-shaped adjustment member 352 maintains the position of cam body 310 in a fashion similar to that of rectangular adjustment member 352 described above.

FIG. 12 illustrates a perspective view of the embodiment of cam adjustment assembly 300 of FIG. 11 as assembled. As described above, cam retainer 302 is fixedly attached to cam body 310. Shaft 330 is fixedly attached between cam body 310 and adjustment member 352. In its assembled form, cam adjustment assembly 300 resides at the anterior end of cage body 100 with cam body 310 positioned within open space 170 between first and second anterior end portions 122, 142, respectively. Cam retainer 302 resides outside of cage body 100 at its anterior end 110 and, acting as a stop or washer, maintains the longitudinal position of cam body 310 in open region 170. Cam retainer 302 prevents movement in the posterior direction of cam adjustment assembly 300 because it contacts anterior end 110 of cage body 100. Similarly, adjustment member 352 prevents movement in the anterior direction of cam adjustment assembly 300 by contact with first end wall 124 and/or second end wall 144 at the anterior end 100 of cage body 100.

FIG. 13 illustrates an alternate embodiment of cam body 310 with one or more cam adjusting features 350. In this embodiment, cam adjusting feature 350 is cam body structure 322. Cam body structure 322 enables the user to engage cam body 310 and rotate it within hollow region 170 of cage body 100. Cam body structure 322 includes, for example, a slot, multiple slots, a star, a square, or another shape that is machined, debossed, or otherwise formed in or on posterior face 312. One cam body structure 322 has two bisecting slots where the center of each slot may be deeper than the edge portion as in a Phillips head screw. Alternately, cam body structure 322 protrudes from, is embossed or formed on, or is attached to posterior face 312 of cam body 310. Examples of a protruding cam body structure 322 include but are not limited to square or hexagonal protrusions that may be engaged with a tool, such as socket wrench or spanner

When cam body structure 322 is formed in cam body 310 as illustrated in FIG. 13, for example, anterior end 110 of cage body 100 preferably has a slot-like open region 170 that retains cam body 310 without the need for cam retainer 302. For example, cam body 310 is sized so that first end wall 124, additional first end wall 124′, second end wall 144, and additional second end wall 144′ retain cam body 310 within open region 170 whether cage body 100 is in a collapsed position or an expanded position. One example of such a slot-like open region 170 is discussed above and illustrated in FIG. 9. In embodiments where open region 170 passes completely through first end portion 122 and second end portion 142, cam retainer 302 on each side of cam body 310 must be used to retain cam body 310 within open region 170. Alternatively, cam body 310 may have a lip or ledge along one or both outer peripheral edges 311 that extends sufficiently to retain cam body 310 within open region 170 even when anterior end 110 of cage body is in a fully expanded configuration. It is contemplated that if only one outer peripheral edge 311 includes the lip or ledge, then a cam retainer 302 would be required.

In use, fusion cage 10 is positioned between adjacent vertebrae of a patient where the vertebrae are accessed posteriorly (i.e., from the patient's back). Cage 10 is positioned so that anterior end 110 of cage body 100 may be subsequently expanded. After cage body 100 is positioned in its collapsed configuration between adjacent vertebrae, the user then inserts an adjustment tool through access opening 162 to cam adjustor assembly 300. By engaging engagement feature 322 of cam body 310, such as a socket placed over cam adjustment member 352, the user rotates adjustment member 352 to cause cam body 310 to also rotate within open region 170 between first anterior end portion 122 and second anterior end portion 142.

Because cam body 310 is generally oblong, its rotation causes anterior end of cage body 100 to expand and increasing the distance between first anterior end portion 122 and second anterior end portion 142. As cam body 310 further rotates, first end 316 and/or second end 318 of cam body 310 occupy first locking notch 136 and second locking notch 156, respectively. Pressure exerted on anterior cage body 100 from anterior portions of the patient's vertebrae bias cage towards its collapsed position, which prevents first end 316 and second end 318 of cam body 310 from freely rotating out of first locking notch 136 and second locking notch 156, respectively. Cage may also be constructed of resilient materials that also bias cage towards its collapsed configuration. Fusion cage 10 may be positioned between adjacent vertebrae by accessing the vertebrae posteriorly where the space between adjacent vertebrae is smaller than the corresponding space between anterior portions of the vertebrae. Fusion cage 10 may subsequently be expanded at its anterior end 110 to provide a snug fit between the vertebrae when the patient's spine is positioned to have the desired lordosis.

Although the preferred embodiments of the present invention have been described herein, the above description is merely illustrative. Further modification of the invention herein disclosed will occur to those skilled in the respective arts and all such modifications are deemed to be within the scope of the invention as defined by the appended claims.

What is claimed is:

1. An expandable fusion cage comprising:
an elongated cage body with a central hollow region, the cage body comprising:
a posterior end having a posterior opening therethrough in communication with the central hollow region;
an anterior end having a first anterior end portion and a second anterior end portion;
a first longitudinal member extending between the posterior end and the first anterior end portion;
a second longitudinal member spaced from and substantially parallel to the first longitudinal member and extending between the posterior end and the second anterior end portion; and
a cam body having a cam surface on a periphery of the cam body and a cam adjusting feature facing the central hollow region, the cam body positioned between the first anterior end portion and the second anterior end portion, the cam body adapted to rotatably engage the cam surface with the first anterior end portion and the second anterior end portion to change the orientation of the anterior end of the cage body between a collapsed configuration and an expanded configuration.

2. The expandable fusion cage of claim 1 wherein the cam body is substantially oblong.

3. The expandable fusion cage of claim 1 wherein the cam adjusting feature is selected from the group consisting of a cam body structure and a cam adjustment member.

4. The expandable fusion cage of claim 3 wherein the cam adjustment member is rectangularly-shaped and has a shaft, wherein the shaft extends substantially parallel to a central longitudinal axis of the elongated cage body and is fixedly attached between the cam body and the cam adjustment member.

5. The expandable fusion cage of claim 1 wherein the first anterior end portion and the second anterior end portion define an open region therebetween, wherein the open region is shaped and configured for rotatable interaction with the cam body.

6. The expandable fusion cage of claim 5 wherein the open region further comprises at least one locking notch configured to receive an end of the cam body.

7. The expandable fusion cage of claim 5 wherein at least one of the first anterior end portion and the second anterior end portion further includes an end wall adjacent to the open region and extending substantially perpendicular to the central longitudinal axis.

8. The expandable fusion cage of claim 6 wherein the cam body further includes a cam retainer configured to retain the cam body between the first anterior end portion and the second anterior end portion.

9. The expandable fusion cage of claim 1 wherein at least one of the first elongated member and the second elongated member includes a pair of spaced-apart members.

10. The expandable fusion cage of claim 1 wherein at least one of the first longitudinal member and the second longitudinal member is hingedly attached to the posterior end.

11. The expandable fusion cage of claim 1 wherein the first longitudinal member and the second longitudinal member are fixedly attached to the posterior end and wherein one of the posterior end or a junction between the posterior end and each of the first longitudinal member and the second longitudinal member is bendable to permit the cage to convert from a collapsed configuration to an expanded configuration upon rotation of the cam body.

12. The expandable fusion cage of claim 1 wherein the cage body is made of a biocompatible material.

13. The expandable fusion cage of claim 12 wherein the biocompatible material is selected from the group consisting of stainless steel, cobalt-chromium-molybdenum alloys, titanium, carbon-reinforced polymers, shape memory alloys, and nylon.

14. The expandable fusion cage of claim 13 wherein the carbon-reinforce polymers include alloys of copper and zinc, nickel titanium, and silver and cadmium.
